(19) United States
(12) Patent Application Publication
(10) Pub. No.: US 2023/0282357 A1
(43) Pub. Date: Sep. 7, 2023

(54) ACUTE LIVER PATIENT MANAGEMENT

(71) Applicant: MERIDIAN BIOSCIENCE ISRAEL LTD., Modi'in (IL)

(72) Inventors: Gil GUGGENHEIM, Jerusalem (IL); Ofer SCHLESINGER, Rehovot (IL); Avraham HERSHKOWITZ, Nof-Ayalon (IL); Ilan BEN OREN, Modiin (IL); Raphael WERNER, Kohav-Yair (IL)

(21) Appl. No.: 17/926,741

(22) PCT Filed: May 27, 2021
(86) PCT No.: PCT/IL2021/050630

§ 371 Date: Nov. 21, 2022

Related U.S. Application Data
(60) Provisional application No. 63/034,468, filed on Jun. 4, 2020.

(51) Int. Cl. G16H 50/30; A61B 5/08; A61B 5/00; A61J 15/00

(52) U.S. Cl. CPC G16H 50/30; A61B 5/082; A61B 5/4244; A61J 15/0003

(57) ABSTRACT

A method for predicting transplant-free survival of a patient suffering from acute liver failure (ALF) or acute liver injury (ALI), the method including: performing a ¹³C-methacetin breath test which includes administering ¹³C labelled methacetin to the patient; measuring exhaled breath to evaluate changes in a ¹³CO₂ to ¹²CO₂ ratio for a predetermined time after the administering; calculating a PDR-peak value of the patient; and predicting the patient as having high chances of transplant-free survival if the calculated PDR-peak value is above a threshold value or as having low chances of transplant-free survival if the PDR-peak value is below threshold value.

### FIELD OF TECHNOLOGY

The present application generally relates to predicting probability of transplant-free survival of a patient suffering from acute liver failure (ALF) or acute liver injury (ALI), using non-invasive monitoring.

### BACKGROUND

Acute liver disorders including severe acute liver disease, acute exacerbation of chronic liver disease and fulminant hepatic failure (FHF) constitute the major causes of morbidity and mortality from liver disease. Decision making in the treatment of patients with severe acute liver disease focuses on early identification of patients that require liver transplantation (Blei, AT. 2005; Liver Transpl., 11 S-30-4.).

The overall transplant-free survival of patients with ALF is approximately 50% (O′Grady, J. 2014; Journal of Hepatology, vol. 60 j 663-670) (Koch, DG. 2016; Clin Gastroenterol Hepatol., 14(8): 1199-1206.e2). Given the significant morbidity and mortality associated with acute liver disease and FHF, there is considerable urgency for the early assessment of patients’ clinical situations and disease severity. Early assessment affects patient placement (intensive care unit versus ward), initiation of supportive therapies and listing for liver transplantation.

Currently, identification is based on several clinical and laboratory parameters, including clinical assessments, serum liver enzymes, synthetic tests and serum ammonia levels, which are time consuming and often lack accuracy in assessing liver function (Blei, ibid.).

Thus, it is often problematic to make decisions concerning the medical treatment and timing of transplantation

A therapeutic dilemma arises from the need to provide expedient transplants to patients with a failing liver while avoiding unnecessary transplantations in patients who are likely to recover spontaneously.

There is therefore an unmet need for a non-invasive, point-of-care monitoring tool which enables fast and accurate predicting of an acute liver failure (ALF) or acute liver injury (ALI) patient’s chances of transplant-free survival.

### SUMMARY OF THE INVENTION

Aspects of the disclosure, in some embodiments thereof, relate to method(s) for predicting transplant-free survival of a patient suffering from acute liver failure (ALF) or acute liver injury (ALI).

Advantageously, the method may assist in prioritizing the allocation of liver transplants by identifying patients having high risk of death. According to some embodiments, the method may enable identifying patients having a risk of lethality and in need of an urgent liver transplantation, in 48 h or less after admittance (or other treatment setting such as an ambulance) and/or in 48 h or less after an acute liver event. It is known that 48-hours survival is of uttermost criticality for overall survival. Therefore, the ability to predict which of the patient will have less chances of 48 h survival and therefore being in need of an urgent transplant is of furthermost importance. On the other hand, patients with high chances of 48 h survival may avoid getting a transplant, which significantly reduces their long term suffering a well as avoids “waste” of transplants.

Advantageously, the method disclosed herein may enable identifying patients having a risk of lethality and in need of an urgent liver transplantation, based on a single, non-invasive breath test.

As a further advantage, the hereindisclosed method takes into consideration the etiology of the patient. It is for example know, that patients suffering from acute liver failure caused by Acetaminophen poisoning have higher chances of transplant-free survival. Accordingly, a different threshold is applied for patients suffering from acute liver failure caused by Acetaminophen poisoning than patients with an etiology different than Acetaminophen poisoning.

Predicting the patient as having high chances of transplant-free survival if the calculated PDR-peak value is above a threshold value or as having low chances of transplant-free survival if the PDR-peak value is below the threshold value, wherein the threshold value depends on the patient’s etiology and wherein if the etiology is Acetaminophen poisoning, the threshold value is higher than for etiologies other than Acetaminophen poisoning.

According to some embodiments, there is provided a method for predicting chances of transplant-free survival of a patient suffering from acute liver failure (ALF) or acute liver injury (ALI), the method by performing ¹³C-methacetin breath test including administering ¹³C-methacetin to the patient; measuring exhaled ¹³CO₂ and ¹²CO₂ for a predetermined time after the administering; calculating a PDR-peak value of the patient, based on the administered ¹³C-methacetin; and predicting the patient as having high chances of transplant-free survival if the calculated PDR-peak value is above a first threshold value or as having low chances of transplant-free survival if the PDR-peak value is below the predetermined first threshold value.

As used herein, the term “Acute liver failure (ALF)” refers to a loss of liver function that occurs rapidly, typically in days or weeks, in a person who has no pre-existing liver disease. Typically, ALF manifests with abnormal liver functions with impaired coagulation (INR >1.5) and signs of encephalopathy. As used herein, the term “acute liver injury (ALI)” refers to patients with coagulopathy and no signs of encephalopathy.

According to some embodiments, the ¹³C-methacetin breath test includes a single (no more than one) ¹³C-methacetin breath test. According to some embodiments, the ¹³C-methacetin breath test us performed within 48 hours after admittance. According to some embodiments, the ¹³C-methacetin breath test us performed within 24 hours after admittance. According to some embodiments, the predicting of transplant-free survival chances is based on the ¹³C-methacetin breath test performed within 24 or 48 hours after admittance only.

According to some embodiments, the first threshold value is equal to or less than 10%/h, equal to or less than 7%/h, equal to or less than 5%/h or equal to or less than 2.5%/h. Each possibility is separate embodiment.

According to some embodiments, the method further comprises obtaining the etiology of the patient and setting the threshold value based on the obtained etiology. According to some embodiments, the etiology of the patient comprises whether or not the acute liver failure/injury is drug induced. According to some embodiments, the etiology of the patient comprises whether or not the acute liver failure/injury is due to Acetaminophen poisoning. According to some embodiments, the threshold value is in a range of about 7%/h to about 10%/h for patients with etiology of Acetaminophen poisoning. According to some embodiments, the threshold value is equal to or less than less than about 5%/h if the etiology of the patient is other than Acetaminophen poisoning.

According to some embodiments, the method further comprises ensuring fasting of the patients for at least 5 hours prior to the breath test.

According to some embodiments, the patient has undergone stomach cleansing prior to the breath test.

According to some embodiments, the predetermined breath testing time is up to 60 minutes.

According to some embodiments, the method further comprises recommending the patient for emergency transplantation if the patient is identified as having low chances of transplant-free survival.

According to some embodiments, the transplant-free survival predicted is a 48-hour transplant-free survival. According to some embodiments, the transplant-free survival predicted is a 5-day transplant-free survival.

According to some embodiments, the patient is a ventilated patient and the ¹³C-methacetin is administered to the patient through a naso-gastral or oro-gastral feeding tube. According to some embodiments, the patient is unconscious.

According to some embodiments, the consumption of allopurinol, carbamazepine, cimetidine, ciprofloxacin, daidzein, disulfiram, echinacea, enoxacin, fluvoxamine, methoxsalen, mexilitene, montelukast, norfloxacin, phenylpropanolamine, phenytoin, propafenone, rifampin, terbinafine, ticlodipine, thiabendazole, verapamil, zileuton and/or oral contraceptives is discontinued at least 48 h before the breath test. Each possibility is a separate embodiment.

According to some embodiments, the subject is administered vasopressors, acyclovir, famotidine and/or statins before and/or during the breath test.

According to some embodiments, the method further comprises predicting chances of a long-term transplant-free recovery. According to some embodiments, the predicting chances of long-term transplant-free recovery of the patient comprises performing at least one additional ¹³C-methacetin breath test; and computing a trend in the PDR-peak value based on the breath test performed within 48 hours after admittance and at least one additional breath test. According to some embodiments, at least one additional breath test is performed 2-4 days after admittance or 1-3 days after the breath test performed within 48 hours after admittance. According to some embodiments, the long-term transplant-free survival comprises at least 20 days of transplant-free survival. According to some embodiments, the patient is predicted as having high chances of long-term transplant-free survival if the computed trend indicates at least a 10% increase in the PDR-peak value between the breath test performed within 48 hours after admittance and a predetermined one of the at least one additional breath tests.

According to some embodiments, there is provided a system for predicting chances of transplant-free survival of a patient suffering from acute liver failure (ALF) or acute liver injury (ALI), the system comprising a processing logic configured to: obtain exhaled ¹³CO₂ and ¹²CO₂ levels measured in the patient a predetermined time after he/she has been administered ¹³C-methacetin; calculate a PDR-peak value of the patient and determine the patient as having high chances of transplant-free survival if the calculated PDR-peak value is below a first threshold value or as having low chances of transplant-free survival if the PDR-peak value is above the predetermined first threshold value.

According to some embodiments, the ¹³C-methacetin breath test includes a single (no more than one) ¹³C-methacetin breath test. According to some embodiments, the ¹³C-methacetin breath test is performed within 48 hours after admittance. According to some embodiments, the ¹³C-methacetin breath test is performed within 24 hours after admittance. According to some embodiments, the predicting of transplant-free survival chances is based on the ¹³C-methacetin breath test performed within 24 or 48 hours after admittance only.

According to some embodiments, the first threshold value is equal to or less than 10%/h, equal to or less than 7%/h, equal to or less than 5%/h or equal to or less than 2.5%/h. Each possibility is a separate embodiment.

According to some embodiments, the processing logic is further configured to obtain the etiology of the patient and to set the threshold value based on the obtained etiology. According to some embodiments, the etiology of the patient comprises whether or not the acute liver failure/injury is drug induced. According to some embodiments, the etiology of the patient comprises whether or not the acute liver failure/injury is due to Acetaminophen poisoning. According to some embodiments, the threshold value set is in a range of about 7%/h to about 10%/h if the patient’s etiology is Acetaminophen poisoning. According to some embodiments, the threshold value set is equal to or less than less than about 5%/h if the etiology of the patient is other than Acetaminophen poisoning.

According to some embodiments, the predetermined breath testing time is up to 60 minutes.

According to some embodiments, the processing logic is further configured to provide a recommendation whether or not the patient be allocated for emergency transplantation based on the determined chances of transplant-free survival.

According to some embodiments, the transplant-free survival predicted is a 48-hour transplant-free survival. According to some embodiments, the transplant-free survival predicted is a 5-day transplant-free survival.

According to some embodiments, the patient is a ventilated. According to some embodiments, the patient is unconscious.

According to some embodiments, the consumption of allopurinol, carbamazepine, cimetidine, ciprofloxacin, daidzein, disulfiram, echinacea, enoxacin, fluvoxamine, methoxsalen, mexilitene, montelukast, norfloxacin, phenylpropanolamine, phenytoin, propafenone, rifampin, terbinafine, ticlodipine, thiabendazole, verapamil, zileuton and/or oral contraceptives is discontinued at least 48 h before the breath test. Each possibility is a separate embodiment.

According to some embodiments, the subject is administered vasopressors, acyclovir, famotidine and/or statins before and/or during the breath test.

According to some embodiments, the processing logic is further configured to predict chances of a long-term transplant-free recovery. According to some embodiments, predicting chances of long-term transplant-free recovery of the patient is based on an additional ¹³C-methacetin breath test performed on the patient. According to some embodiments, predicting chances of long-term transplant-free recovery comprises computing a trend in the PDR-peak value based on the breath test performed within 48 hours after admittance and at least one additional breath test. According to some embodiments, at least one additional breath test is performed 2-4 days after admittance or 1-3 days after the breath test performed within 48 hours after admittance. According to some embodiments, the long-term transplant-free survival comprises at least 20 days of transplant-free survival. According to some embodiments, the patient is predicted as having high chances of long-term transplant-free survival if the computed trend indicates at least a 10% increase in the PDR-peak value between the breath test performed within 48 hours after admittance and a predetermined one of at least one additional breath tests.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE FIGURES

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show details of an embodiment in more detail than is necessary for a fundamental understanding of the teachings of the disclosure.

FIG. 1 is a flowchart of a method for predicting 48 h transplant-free survival of a patient suffering from acute liver failure (ALF) or acute liver injury (ALI) using a ¹³C-methacetin breath test, according to some embodiments;

FIG. 2 is a flowchart of a method for predicting transplant-free survival of a patient suffering from acute liver failure (ALF) or acute liver injury (ALI) based on a single ¹³C-methacetin breath test, according to some embodiments;

FIG. 3 is a flowchart of a method for predicting transplant-free survival of a patient suffering from acute liver failure (ALF) or acute liver injury (ALI), while taking into consideration patient etiology, according to some embodiments;

FIG. 4 is a flowchart of a method for prioritizing acute liver failure (ALF) or acute liver injury (ALI) patients for allocation of liver transplants, according to some embodiments;

FIG. 5 shows average PDR-peak values by outcome and ¹³C-methacetin breath test administration day of transplant-free survivors (TFS) of acute liver failure (red/right bars) and non-TFS (blue/ left bars).

FIG. 6 shows serial PDR-peak results in an ALF drug-induced liver injury (DILI) patient who survived without transplant (stippled line) and an ALI DILI patient who required liver transplantation (full line).

FIG. 7 shows the AUROC for PDR-peak to predict 21-day transplant free survival in ALF and ALI patients.

FIG. 8 shows the AUROC for PDR-peak to predict 21-day transplant free survival in ALF patients.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

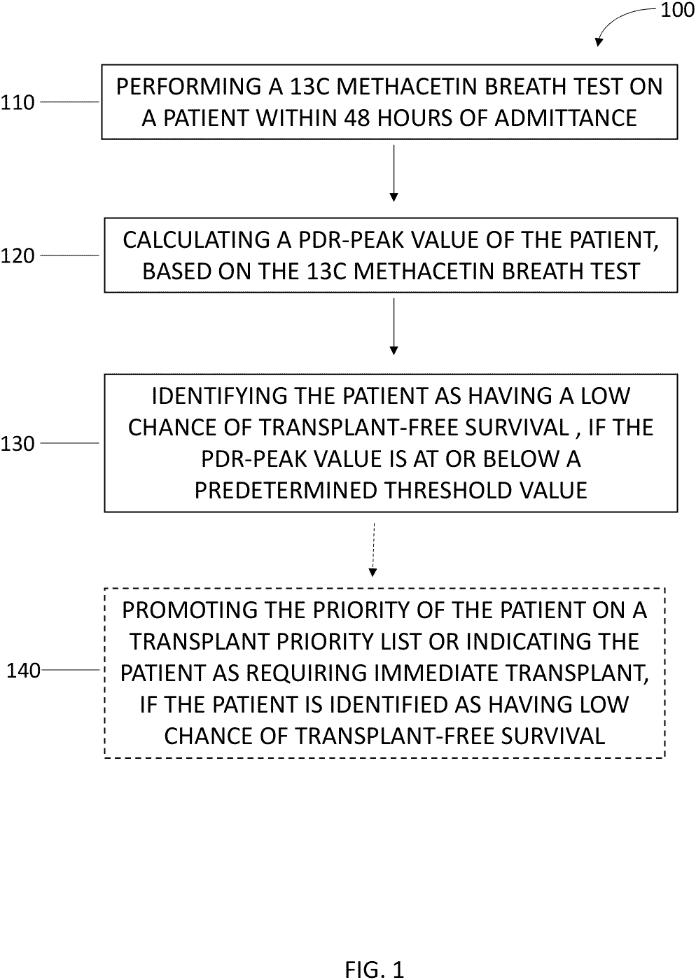

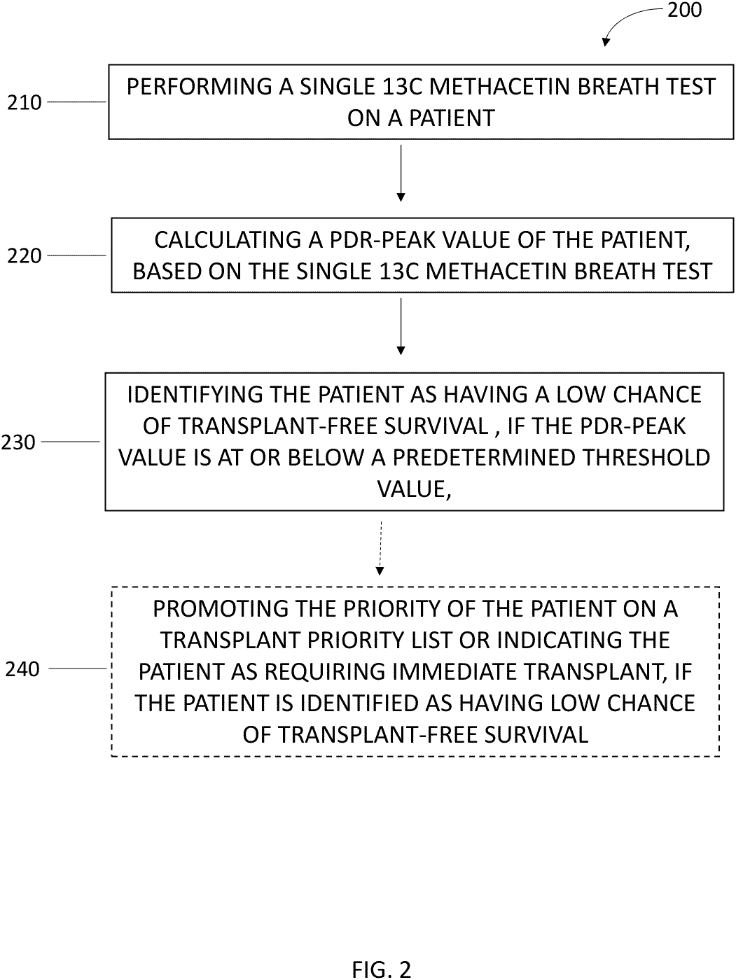

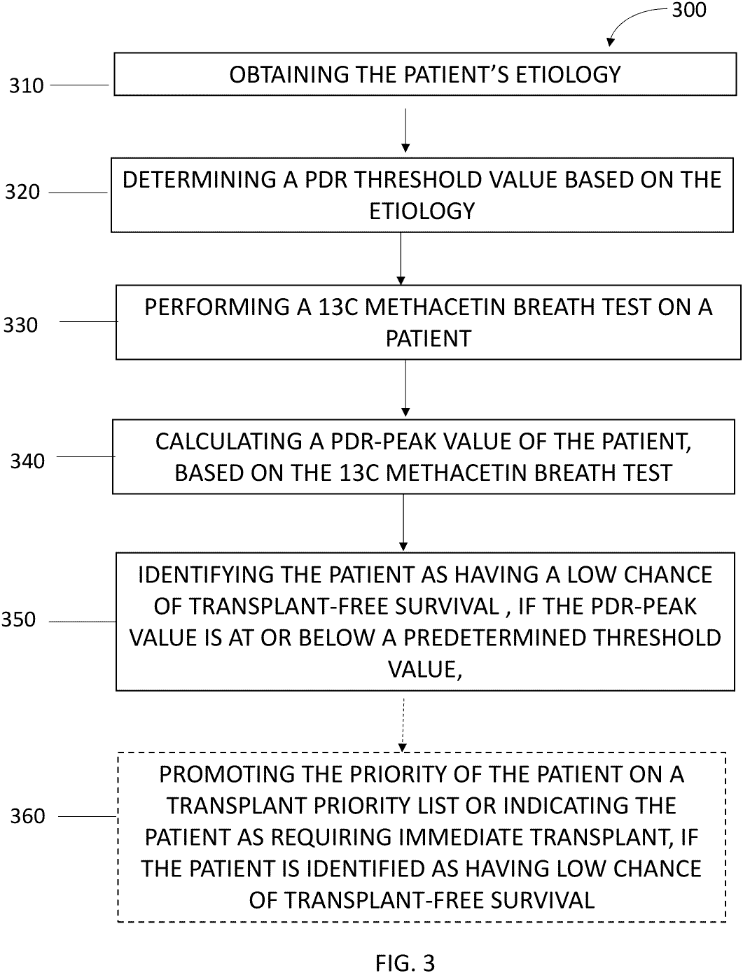

FIG. 1-FIG. 4 show flowchart of the herein disclosed methods. The methods are shown as being separate. However, it is understood that they may also be combined. For example, the method disclosed in FIG. 1 which is directed to performing a breath test within 48 h of admittance of the patient may be combined with the method disclosed in FIG. 2 which is directed to a method of predicting transplant-free survival of a patient suffering from acute liver failure (ALF) or acute liver injury (ALI) based on a single breath test and/or with FIG. 3 which is directed to predicting transplant-free survival of a patient suffering from acute liver failure (ALF) or acute liver injury (ALI), while taking into consideration the patient’s etiology. It is also understood that, at least with regards to some steps, the order of the steps may be changed. For example, in FIG. 3, the etiology of the patient may be obtained before or after the breath test, but before predicting the transplant-free survival of the patient. It is also understood that the method may include additional steps, such as, but not limited to, ensuring fasting of the patients for at least 5 hours prior to the breath test, cleansing of the patient’s stomach prior to the breath test and/or discontinuing administration of certain medications before the breath test, etc.

Reference is now made to FIG. 1 which is a flowchart of a method 100 for predicting 48 h transplant-free survival of a patient suffering from acute liver failure (ALF) or acute liver injury (ALI) using a ¹³C-methacetin breath test.

In step 110 a ¹³C-methacetin breath test is performed within 48 hours of admittance on the patient. As used herein, the term “within 48 h of admittance” may refer to 48 h from being hospitalized, 48 h from the acute liver failure/injury (e.g. for already hospitalized patients) or within 48 h from the patient being in a setting allowing the breath test from being performed (within 48 h from the earliest possible). Based on the ¹³C-methacetin breath test measurements, a PDR-peak value of the patient is calculated (step 120). In step 130 the patient is identified as having high-risk of lethality (i.e. low chance of transplant-free survival), if the PDR-peak value is at or below a predetermined threshold value. According to some embodiments, the predetermined threshold value is at or below a PDR-peak value in the range of 2.5%/h-10%/h. The actual threshold value chosen is a question of risk-management and preferences. It is understood that when the threshold is set closer to 10%/h (e.g. 9%/h) the risk of wrongful exclusion of a high-risk patient is diminished however many non-high-risk patients may be included, thus reducing the specificity of the method. On the other hand, if a threshold close to 2.5% is chosen (e.g. 3.5%) the risk of wrongful exclusion of a high-risk patient is increased, however the certainty of patients identified as being high-risk actually being high risk significantly improved.

Optionally, method 100 may further include a step 140 of promoting the priority of the patient on a transplant priority list and/or indicating the patient as need of immediate transplant, if the patient is identified as having low chance of transplant-free survival.

Reference is now made to FIG. 2 which is a flowchart of a method 200 for predicting transplant-free survival of a patient suffering from acute liver failure (ALF) or acute liver injury (ALI) based on a single ¹³C-methacetin breath test.

In step 210 a single ¹³C-methacetin breath test is performed on the patient followed by a calculation of at least a PDR-peak value of the patient, based on the ¹³C-methacetin breath test (step 220). In step 230 the patient is identified as having high-risk of lethality (i.e. low chance of transplant-free survival), if the PDR-peak value is at or below a predetermined threshold value. According to some embodiments, the predetermined threshold value is at or below a PDR-peak value in the range of 2.5%/h-10%/h. The actual threshold value chosen is a question of risk-management and preferences. It is understood that when the threshold is set closer to 10%/h (e.g. 9%/h) the risk of wrongful exclusion of a high-risk patient is diminished however many non-high-risk patients may be included, thus reducing the specificity of the method. On the other hand, if a threshold close to 2.5% is chosen (e.g. 3.5%) the risk of wrongful exclusion of a high-risk patient is increased, however the certainty of patients identified as being high-risk actually being high risk significantly improved. As used herein, the term “single ¹³C-methacetin breath test” refers to the prediction being made solely based on a single (i.e. no more than one) ¹³C-methacetin breath test. According to some embodiments, the prediction is based only on the single ¹³C-methacetin breath test.

Optionally, method 200 may further include a step 240 of promoting the priority of the patient on a transplant priority list and/or indicating the patient as need of immediate transplant, if the patient is identified as having low chance of transplant-free survival.

Reference is now made to FIG. 3 which is a flowchart of a method 300 for predicting transplant-free survival of a patient suffering from acute liver failure (ALF) or acute liver injury (ALI) using a methacetin breath test, while taking into consideration patient etiology.

In step 310, the etiology of the patient is obtained. According to some embodiments, the etiology comprises whether or not the ALF/ALI is caused by Acetaminophen poisoning. According to some embodiments, the etiology may further include the patient’s age, sex, weight and/or medical history. Each possibility is a separate embodiment. According to some embodiments, the medical history comprises whether or not the patient has experienced prior liver problems (e.g. prior ALF/ALI). According to some embodiments, the medical history comprises whether or not the patient has other comorbidities (e.g. cardiac issues, diabetes etc.).

In step 320, a PDR-peak threshold value is determined, based on the obtained etiology. According to some embodiments, the PDR-peak threshold value may be higher for patients with ALF due to Acetaminophen poisoning than patients, of whom the ALF is not related to Acetaminophen poisoning. According to some embodiments, the PDR-peak threshold value may be in a range of 7%/h-10%/h for ALF patients with Acetaminophen poisoning and 2.5%/h-7%/h for non-Acetaminophen poisoning ALF patients.

In step 330 a ¹³C-methacetin breath test is performed on the patient followed by a calculating of at least a PDR-peak value of the patient, based on the ¹³C-methacetin breath test (step 340). In step 230 the patient is identified as having high-risk of lethality (i.e. low chance of transplant-free survival), if the PDR-peak value is at or below a predetermined threshold value.

Optionally, method 300 may further include a step 360 of promoting the priority of the patient on a transplant priority list and/or indicating the patient as need of immediate transplant, if the patient is identified as having low chance of transplant-free survival.

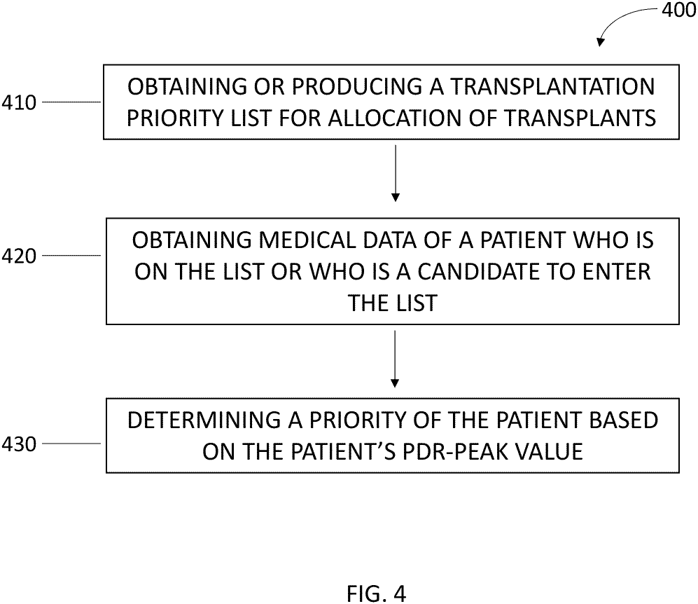

Reference is now made to FIG. 4 which is a flowchart of a method 400 for prioritizing ALF/ALI patients for allocation of liver transplant.

In step 410 a transplantation priority list for allocation of transplants is obtained or being generated; in step 420 medical data of a patient who suffers from ALF/ALI who is on the transplantation priority list or who is a candidate to enter the transplantation priority list are obtained. The data include at least ¹³C-methacetin breath test results of the patients. According to some embodiments, the ¹³C-methacetin breath test results may be “raw” data in which case step 420 includes calculating a PDR-peak value of the patient, based on the raw ¹³C-methacetin breath test data. Alternatively, the PDR-peak value of the patient may be precalculated and directly obtained.

In step 430 a priority of the patient may be determined (number on list) based on the patient’s PDR-peak value. That is, if the PDR-peak value is at or below a predetermined threshold value, the patient may receive a higher priority on the transplant priority list relative to patients suffering from ALF or ALI, but who have a PDR-peak value above the predetermined threshold value.

According to some embodiments, the predetermined threshold value is at or below a PDR-peak value in the range of 2.5%/h-10%/h. The actual threshold value chosen is a question of risk-management and preferences. It is understood that when the threshold is set closer to 10%/h (e.g. 9%/h) the risk of wrongful exclusion of a high-risk patient (i.e. providing him/her with a lower than “deserved” priority) is diminished; however many non-high-risk patients may be included (thus providing them with a higher than “deserved” priority). On the other hand, if a threshold close to 2.5% is chosen (e.g. 3.5%) the risk of wrongful exclusion of a high-risk patient is increased; however, the certainty of patients identified as being high-risk actually being high risk significantly improved.

The following examples are presented in order to illustrate in more details some embodiments of the invention. They should in no way be construed, however, as limiting the broad scope of the invention. One skilled in the art can readily devise many variations and modifications of the principles disclosed herein without departing from the scope of the invention.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising”, when used in this specification, specify the presence of stated features, integers, steps, operations, elements, or components, but do not preclude or rule out the presence or addition of one or more other features, integers, steps, operations, elements, components, or groups thereof. Unless otherwise defined, all technical and scientific terms used herein have the same meaning as commonly understood by one of ordinary skill in the art to which this invention belongs.

Unless specifically stated otherwise, as apparent from the following discussions, it is appreciated that throughout the specification discussions utilizing terms such as “processing”, “computing”, “calculating”, “determining”, “estimating”, or the like, refer to the action and/or processes of a computer or computing system, or similar electronic computing device, that manipulate and/or transform data represented as physical, such as electronic, quantities within the computing system’s registers and/or memories into other data similarly represented as physical quantities within the computing system’s memories, registers or other such information storage, transmission or display devices.

Embodiments of the present invention may include apparatus for performing the operations herein. This apparatus may be specially constructed for the desired purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

The invention may be described in the general context of computer-executable instructions, such as program modules, being executed by a computer. Generally, program modules include routines, programs, objects, components, data structures, and so forth, which perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

### EXAMPLES

### Example 1 - Use of ¹³C-Methacetin Breath Test for Determining Acute Liver Failure or Acute Liver Injury Severances: Clinical Experience and Correlations

A ¹³C-methacetin breath test (MBT), which assays CYP1A2 metabolism, for assessing functional liver capacity and its changes over time, was utilized to predict transplant-free survival.

Method: Eligible patients with ALF or non-acetaminophen (APAP) ALI underwent ¹³C-MBT at day 1, 2, 3, 5 and 7 after enrollment, unless discharged, transplanted or died. 75 mg of ¹³C-methacetin was administered orally or via a nasogastric tube, and exhaled ¹³CO₂ measured for up to 60 minutes using the BreathID® (Exalenz Bioscience Ltd., Modi’in, Israel). The percent dose recovery (PDR) peak was determined on day 1 or 2 and correlated with 21-day outcome.

Results: 280 subjects were screened for enrollment between 5/2016 and 8/2019 at 11 ALF Study Group clinical sites. Of 76 patients enrolled, 62 had adequate MBT data for analysis. Mean age was 43±15 y, 61% female, 79% were Caucasian, 76% had ALF due to a multitude of etiologies and 24% had non-APAP ALI. At enrollment, 42% had grade 3 or 4 encephalopathy, median bilirubin 7.2 mg/dl and median INR 2.7. By Day 21, 21% had undergone liver transplant and 16% died. There were no serious adverse events amongst the 185 tests performed that were attributable to test device or substrate. None of the first 20 patients enrolled had de-novo or worsening serum cysteine-APAP protein adduct levels on serial testing.

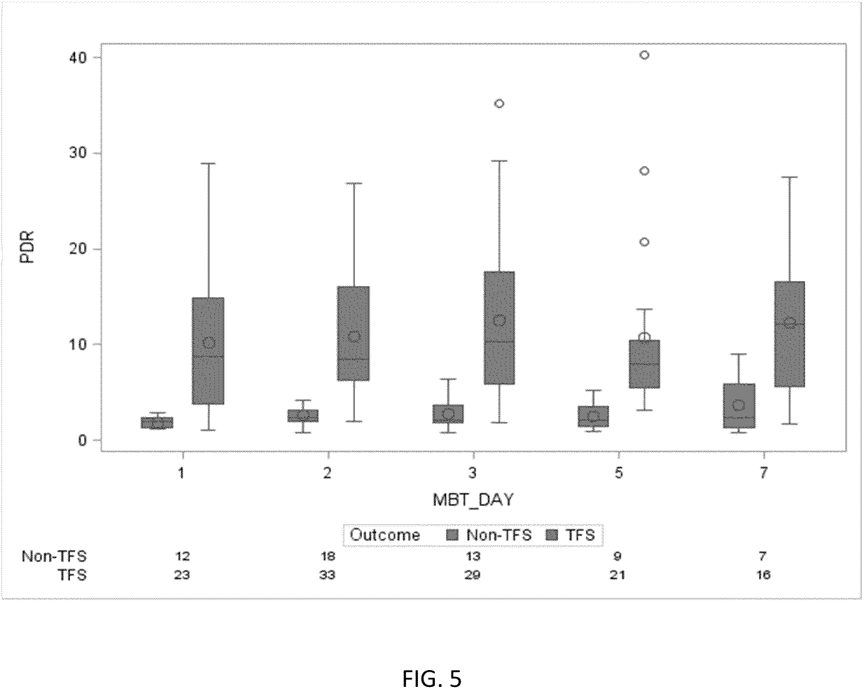

Advantageously, day 1 or 2 mean PDR-peak was significantly higher in transplant-free survivors (TFS) vs non-TFS (9.2 %/h vs 2.3 %/h, p < 0.0001). Furthermore, mean serial PDR-peaks were consistently higher in TFS vs non-TFS, as seen from FIG. 5 which shows the PDR-peak by outcome and ¹³C-methacetin breath test administration day. The PDR-peak values were significantly lower in the non-survivors compared to the transplant free survivors (p < 0.001, repeated measures analysis).

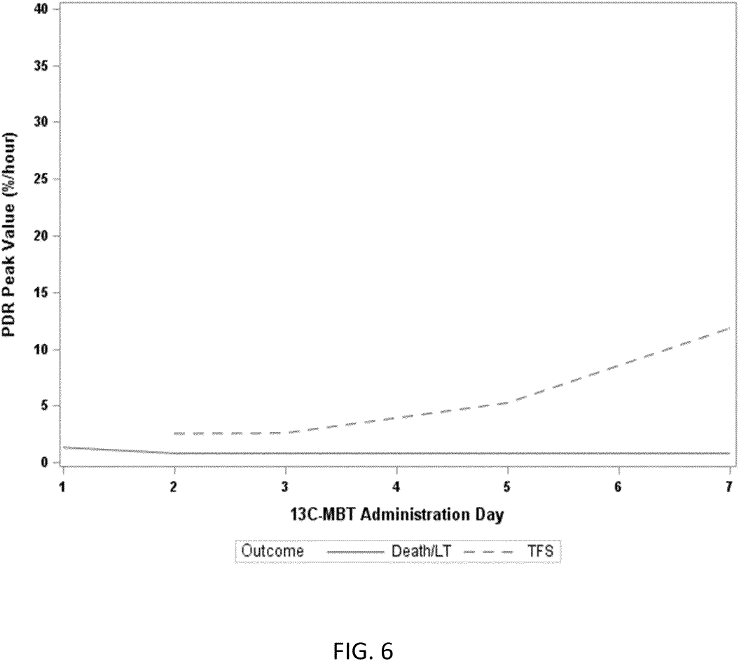

FIG. 6 shows serial PDR-peak results in an ALF drug-induced liver injury (DILI) patient who survived without transplant (stippled line) and an ALI DILI patient who required liver transplantation (full line).

A) A 52-year-old female with presumed DILI due to tizanidine and ropinirole was enrolled with a MELD score of 26 and a total bilirubin of 9.3 mg/dl, INR 2.8, and grade 3 hepatic encephalopathy. She received intravenous N-acetylcysteine on day 1 and was on famotidine starting on day 2, but never on pressors nor intubated. Over time her clinical status improved and by day 7 her hepatic encephalopathy grade reached 1 and she was discharged at day 14. As seen from FIG. 6, her PDR peak results improved from day 2 through day 7 consistent with transplantation free survival.

B) A 53-year-old female with DILI due to clindamycin was enrolled with ALI and no encephalopathy. During follow-up, she rapidly deteriorated and was listed for liver transplantation. She was never on pressors but was intubated on day 6. She eventually underwent liver transplantation on study day 7 and discharged on day 20. As seen from FIG. 6, the PDR peak test results predicted the clinical outcome in the form of a very low PDR-peak during the entire period.

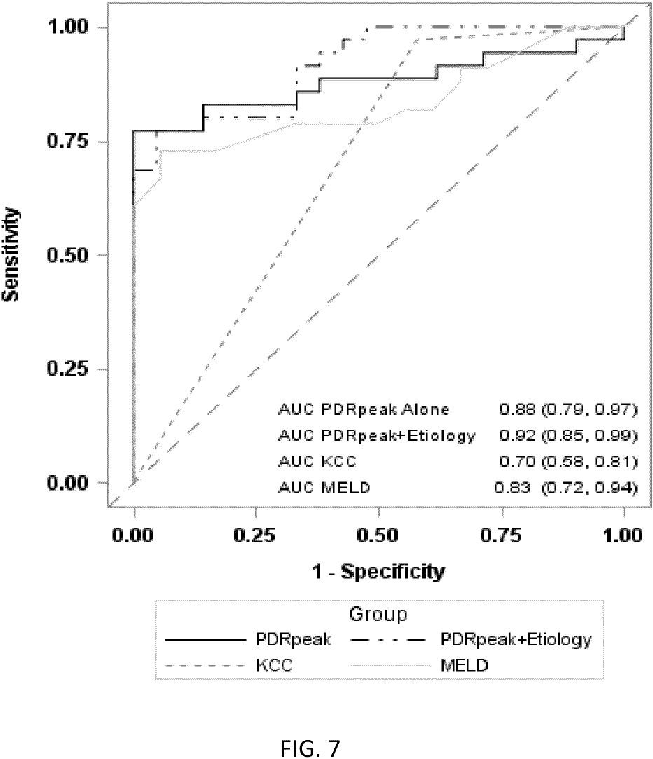

FIG. 7 shows the AUROC of the PDR-peak in prediction of 21-day transplant free survival in the combined ALF or ALI cohort. Amongst 56 ALF and ALI patients with day 1 or 2 measures, the PDR-peak as a predictor of TFS had an AUROC of 0.88 (95% CI 0.79-0.97) which was further improved to 0.92 (95% CI 0.85-0.99) with the inclusion of the etiology of ALI/ ALF. In comparison, the AUROC for 21-day survival of the King’s College criteria (KCC) was 0.70 and day 1 MELD score 0.83, clearly showing the superiority of the herein disclosed MBT test.

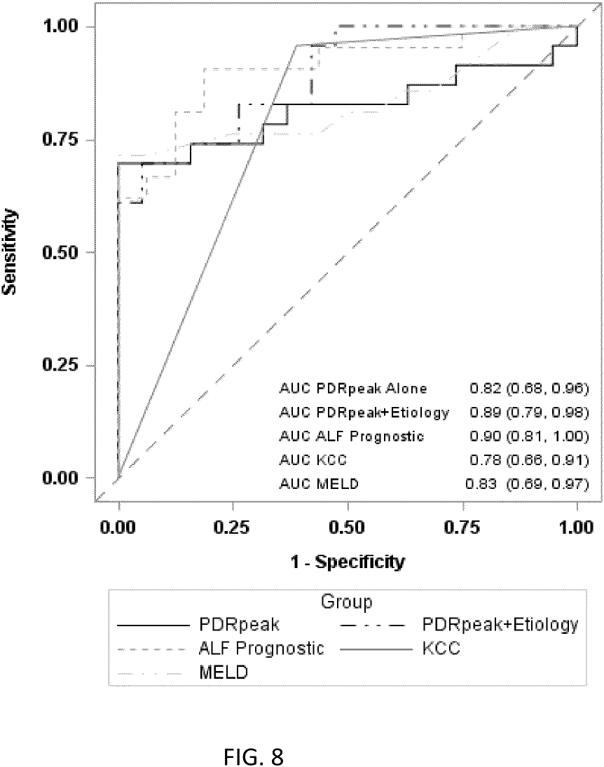

FIG. 8 shows the AUROC of the PDR-peak in prediction of 21-day transplant free survival in the ALF patients alone. Amongst the 42 ALF patients with available data, the AUROC for the Day 1 or 2 PDR-peak values was 0.82 (95% CI 0.68-0.96) which improved to 0.89 (95% CI 0.79-0.98) with inclusion of ALF etiology and to 0.93 when combined with the ALFSG prognostic index. In comparison, the AUROC was 0.78 for KCC and 0.83 for MELD scores, clearly showing the superiority of the herein disclosed MBT test.

Conclusion: The ¹³C MBT is a non-invasive, semi-quantitative hepatic function test that can help identify individuals with ALF or non-APAP ALI who are likely to recover without a transplant. Advantageously, the test allows early prediction in that a single PDR-peak value, obtained at day of enrolment, or the day thereafter, sufficed for predicting transplant-free survival.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1-65. (canceled)

66. A method for predicting transplant-free survival of a patient suffering from acute liver failure (ALF) or acute liver injury (ALI), the method comprising:
performing ¹³C-methacetin breath test by:
administering ¹³C-methacetin to the patient; and
measuring exhaled ¹³CO₂ and ¹²CO₂ for a predetermined time after the administration;
calculating a PDR-peak value of the patient, based on the administered ¹³C-methacetin; and
predicting the patient as having high chances of transplant-free survival if the calculated PDR-peak value is above a first threshold value or as having low chances of transplant-free survival if the PDR-peak value is below the predetermined first threshold value, wherein the ¹³C-methacetin breath test comprises no more than one ¹³C-methacetin breath test.

67. The method of claim 66, wherein the¹³C-methacetin breath test is performed within 48 hours after admittance.

68. The method of claim 66, wherein the method further comprises fasting of the patients for at least 5 hours prior to the breath test.

69. The method of claim 66, wherein the patient has undergone stomach cleansing prior to the breath test.

70. The method of claim 66, wherein the first threshold value is equal to or less than less than 10%/h.

71. The method of claim 66, wherein the transplant-free survival predicted is a 48-hour or a 5-day transplant-free survival.

72. The method of claim 66, wherein the patient is a ventilated patient and wherein the ¹³C-methacetin is administered to the patient through a naso-gastral or oro-gastral feeding tube.

73. The method of claim 66, wherein consumption of allopurinol, carbamazepine, cimetidine, ciprofloxacin, daidzein, disulfiram, echinacea, enoxacin, fluvoxamine, methoxsalen, mexilitene, montelukast, norfloxacin, phenylpropanolamine, phenytoin, propafenone, rifampin, terbinafine, ticlodipine, thiabendazole, verapamil, zileuton and/or oral contraceptives is discontinued at least 48 h before the breath test.

74. A method for predicting chances transplant-free survival of a patient suffering from acute liver failure (ALF) or acute liver injury (ALI), the method comprising:
obtaining information regarding the etiology of the patient
performing a ¹³C-methacetin breath test, the test comprising:
administering ¹³C-methacetin to the patient;
measuring exhaled ¹³CO₂ for a predetermined time after the administering; and
calculating a PDR-peak value of the patient, based on the administered ¹³C-methacetin; and
predicting the patient as having high chances of transplant-free survival if the calculated PDR-peak value is above a threshold value or as having low chances of transplant-free survival if the PDR-peak value is below the threshold value, wherein the threshold value depends on the patient’s etiology and wherein if the etiology is Acetaminophen Poisoning the threshold value is higher than the etiology is other than Acetaminophen Poisoning.

75. The method of claim 74, wherein the ¹³C-methacetin breath test is performed within 48 hours after admittance.

76. The method of claim 74, wherein the method further comprises fasting of the patients for at least 5 hours prior to the breath test.

77. The method of claim 74, wherein the patient has undergone stomach cleansing prior to the breath test.

78. The method of claim 74, wherein if the etiology of the patient is acetaminophen poisoning the threshold value is in a range of about 7%/h to about 10%/h and if the etiology of the patient is other than Acetaminophen poisoning the threshold value is equal to or less than less than about 5%/h.

79. The method of claim 74, wherein the transplant-free survival predicted is a 48-hour or a 5-day transplant-free survival.

80. The method of claim 74, further comprising predicting chances of a long-term transplant-free recovery, wherein the long-term transplant-free survival comprises at least 20 days of transplant-free survival.

81. The method of claim 80, wherein predicting chances of long-term transplant-free recovery of the patient comprises performing at least one additional ¹³C-methacetin breath test; and computing a trend in the PDR-peak value based on the breath test performed within 48 hours after admittance and at least one additional breath test.

82. The method of claim 81, wherein the patient is predicted as having high chances of long-term transplant-free survival if the computed trend indicates at least a 10% increase in the PDR-peak value between the breath test performed within 48 hours after admittance and one of the predetermined additional breath tests.

83. The method of claim 74, wherein the patient is a ventilated patient and wherein the ¹³C-methacetin is administered to the patient through a naso-gastral feeding tube.

84. The method of claim 74, wherein consumption of allopurinol, carbamazepine, cimetidine, ciprofloxacin, daidzein, disulfiram, echinacea, enoxacin, fluvoxamine, methoxsalen, mexilitene, montelukast, norfloxacin, phenylpropanolamine, phenytoin, propafenone, rifampin, terbinafine, ticlodipine, thiabendazole, verapamil, zileuton and/or oral contraceptives is discontinued at least 48 h before the breath test.
